(19) United States
(12) Patent Application Publication
(10) Pub. No.: US 2015/0166567 A1
(43) Pub. Date: Jun. 18, 2015

(54) PROTEASOME ACTIVITY ENHANCING COMPOUNDS

(71) Applicant: Proteostasis Therapeutics, Inc., Cambridge, MA (US)

(72) Inventors: Robert J. Chambers, Charlestown, MA (US); Megan Foley, Cambridge, MA (US); Bradley Tait, Malden, MA (US)

(21) Appl. No.: 14/333,957

(22) Filed: Jul. 17, 2014

Related U.S. Application Data
(63) Continuation of application No. PCT/US2013/022912, filed on Jan. 24, 2013.
(60) Provisional application No. 61/590,606, filed on Jan. 25, 2012.
(60) Provisional application No. 61/739,077, filed on Dec. 19, 2012.

(51) Int. Cl. C07D 495/14

(52) U.S. Cl. CPC C07D 495/14

(57) ABSTRACT

The present invention is directed to compounds having the Formula (I), (Ia) or (Ib), compositions thereof and methods for the treatment of a condition associated with a dysfunction in proteostasis.

### RELATED APPLICATIONS

This application is a continuation of International Application No. PCT/US2013/022912, which designated the United States and was filed on Jan. 24, 2013, published in English, which claims the benefit of U.S. Provisional Application No. 61/590,606 filed Jan. 25, 2012 and U.S. Provisional Application No. 61/739,077 filed Dec. 19, 2012. The entire teachings of the above application are incorporated herein by reference.

### BACKGROUND OF THE INVENTION

Cells normally maintain a balance between protein synthesis, folding, trafficking, aggregation, and degradation, referred to as protein homeostasis, utilizing sensors and networks of pathways [Sitia et al., Nature 426: 891-894, 2003; Ron et al., Nat Rev Mol Cell Biol 8: 519-529, 2007]. The cellular maintenance of protein homeostasis, or proteostasis, refers to controlling the conformation, binding interactions, location and concentration of individual proteins making up the proteome. Protein folding in vivo is accomplished through interactions between the folding polypeptide chain and macromolecular cellular components, including multiple classes of chaperones and folding enzymes, which minimize aggregation [Wiseman et al., Cell 131: 809-821, 2007]. Whether a given protein folds in a certain cell type depends on the distribution, concentration, and subcellular localization of chaperones, folding enzymes, metabolites and the like [Wiseman et al.]. Human loss of function diseases are often the result of a disruption of normal protein homeostasis, typically caused by a mutation in a given protein that compromises its cellular folding, leading to efficient degradation [Cohen et al., Nature 426: 905-909, 2003]. Human gain of function diseases are similarly frequently the result of a disruption in protein homeostasis, such as the accumulation of misfolded proteins, leading to protein aggregation [Balch et al. (2008), Science 319: 916-919].

The proteasome is a large protein complex of multiple subunits which acts as a protease to degrade misfolded proteins. Most proteasome substrates are targeted for degradation by the covalent attachment of ubiquitin moieties which are recognized by the proteasome [Lee et al. (2010), Nature 467(7312): 179-184]. Proteins with longer ubiqutin chains tend to have a stronger association with the proteasome than those with smaller chains [Lee et al. (2010); Proctor et al. (2007), BMC Systems Biology 1: 17]. The length of the ubiquitin chains is modulated, in part, by proteasome-associated deubiquitinating enzymes. One such mammalian deubiquitinating enzyme is Usp14 which has been shown to act as an inhibitor of the proteasome [Lee et al. (2010)].

Both proteasome dysfunction and dysfunction in proteostasis have been implicated in a diverse range of diseases including for example, neurodegenerative disease, metabolic diseases, inflammatory diseases, and cancer. In many such diseases and conditions, the proteasome has decreased ability to degrade misfolded or abnormal proteins, leading to the presence of toxic protein aggregates. In addition, the enhancement of proteasome activity can be therapeutic for any disease characterized by deficient proteasome activity, or deficient activity of other components of the ubiquitin-proteasome pathway including, but not limited to, von Hippel-Lindau disease, spinocerebellar ataxia 1, Angelman syndrome, giant axon neuropathy, inclusion body myopathy with Paget disease of bone and frontotemporal dementia (IBMPFD), and others [Lehman, N. L., (2009), Acta Neuropathologica, 118(3), 329-347; Weihl et al., (2007), Neuromuscular Disorders, 17, 87-87]. Enhancing proteasome activity is also therapeutic for diseases in which proteasome substrates are involved and contribute to pathology, but which do not satisfy a strict definition of proteinopathies. For example, numerous oncoproteins are proteasome substrates and their ability to promote cancer can potentially be attenuated by enhancing proteasome activity. Therefore, there is a need for compounds and pharmaceutical compositions to treat conditions associated with proteostasis dysfunction and/or that provide therapies based on enhancing proteasome activity.

### SUMMARY OF THE INVENTION

The present invention is based, in part, on the discovery that compounds of the invention inhibit Usp14. The present invention is directed to compounds encompassed by the Formulae (I), (Ia) and (Ib), compositions thereof, methods for the treatment of a condition associated with a dysfunction in proteostasis, methods for enhancing proteasome activity and methods for treating cancer or tumor.

In one embodiment, the invention is directed to a compound having the Formula (I):

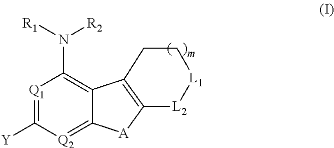

or a pharmaceutically acceptable salt, solvate, clathrate or prodrug thereof; wherein:

Q₁ and Q2 are each independently selected from the group consisting of nitrogen and CR₆ₐ;

A is sulfur, oxygen or NR₅ₐ;

R₁ and R₂ are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₁-C₁₀ alkoxy, optionally substituted C₃-C₁₂ cycloalkyl, optionally substituted C₃-C₁₂ cycloalkenyl, optionally substituted heterocyclic, optionally substituted aryl and optionally substituted heteroaryl; or alternatively, R₁ and R₂ are taken together with the nitrogen atom to which they are attached to form an optionally substituted heterocylic or an optionally substituted heteroaryl;

Y is selected from the group consisting of hydrogen and

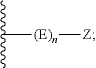

E is C(R₄ₐ)(R_{4b});

Z is selected from the group consisting of hydrogen, optionally substituted C₁-C₁₀ alkyl, optionally substituted C₂-C₁₀ alkenyl, optionally substituted C₂-C₁₀ alkynyl, OR₃, C(O)R₃, C(O)OR₃, C(O)NRₐS(O)₂R₃, OC(O)R₃, C(O)NRₐR_{b}, S(O)₂NRₐR_{b}, S(O)NRₐR_{b}, NRₐS(O)₂R₃, CN, SR₃, S(O)R₃, S(O)₂R₃, P(O)(OR₃)₂, NRₐR_{b}, N(Rₐ)OR₃, NRₐC(O)C(O)R₃, NRₐC(O)R₃, NRₐC(O)NRₐR_{b}, NRₐS(O)₂NRₐR_{b}, optionally substituted C₃-C₁₂ cycloalkyl, optionally substituted C₃-C₁₂ cycloalkenyl, optionally substituted heterocyclic, optionally substituted aryl and optionally substituted heteroaryl;

R₃ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;

Rₐ and R_{b} are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; alternatively, Rₐ and R_{b} are taken together with the nitrogen atom to which they are attached to form an optionally substituted heterocyclic or an optionally substituted heteroaryl;

L₁, L₂, L₃ and L₄ are each independently selected from C(R₆ₐ)(R_{6b}), O, NR_{d}, S, S(O), and SO₂, wherein at least one of L₁, L₂, L₃ and L₄ is O, NR_{d}, S, S(O) and SO₂;

each of R₄ₐ and R_{4b} are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{c}, SR_{c}, NRₐR_{b}, C(O)OR_{c}, NO₂, CN, C(O)R_{c}, C(O)C(O)R_{c}, C(O)NRₐR_{b}, NRₐC(O)R_{c}, NRₐS(O)ₚR_{c}, N(Rₐ)C(O)OR_{c}, NRₐC(O)C(O)R_{c}, NRₐC(O)NRₐR_{b}, NRₐS(O)ₚNRₐR_{b}, S(O)ₚR_{c}, S(O)ₚNRₐR_{b}, OC(O)OR_{c}, and (C═NRₐ)R_{c}; alternatively, R₄ₐ and R_{4b} can be taken together with the carbon atom to which they are attached to form an optionally substituted optionally substituted C₃-C₁₂ cycloalkyl, optionally substituted C₃-C₁₂ cycloalkenyl, optionally substituted heterocyclic, optionally substituted aryl or optionally substituted heteroaryl;

R₅ₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;

each of R₆ₐ and R_{6b} are, at each occurrence, independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{c}, SR_{c}, NRₐR_{b}, C(O)OR_{c}, NO₂, CN, C(O)R_{c}, C(O)C(O)R_{c}, C(O)NRₐR_{b}, C(O)NRₐS(O)₂R₃, NRₐC(O)R_{c}, NRₐS(O)ₚR_{c}, N(Rₐ)C(O)OR_{c}, NRₐC(O)C(O)R_{c}, NRₐC(O)NRₐR_{b}, NRₐS(O)ₚNRₐR_{b}, S(O)ₚR_{c}, S(O)ₚNRₐR_{b}, OC(O)OR_{c}, and (C═NRₐ)R_{c}; alternatively, geminal R₆ₐ and R_{6b} can be taken together with the carbon atom to which they are attached to form a Spiro C₃-C₁₂ cycloalkyl, a spiro C₃-C₁₂ cycloalkenyl, a spiro heterocyclic, a spiro aryl or spiro heteroaryl, each optionally substituted;

each R_{c} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;

each R_{d} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, C(O)R_{c}, C(O)C(O)R_{c}, C(O)OR_{c}, C(O)NRₐR_{b}, S(O)ₚR_{c}, and S(O)ₚNRₐR_{b};

m and n are each independently selected from the group consisting of 0, 1, 2 and 3; and

p is 1 or 2.

In an additional embodiment, the invention is a compound having the Formula (Ia):

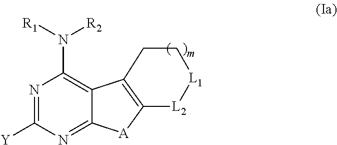

or a pharmaceutically acceptable salt, solvate, clathrate or prodrug thereof; wherein: Y, E, Z, R₁, R₂, R₃, Rₐ, R_{b}, R_{c}, R_{d}, R₄ₐ, R_{4b}, R₅ₐ, R₆ₐ, R_{6b}, A, L₁, L₂, m, n and p are as defined above for Formula (I).

In a further embodiment, the invention is directed to a compound having to Formula (Ib):

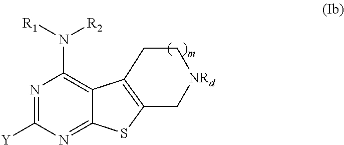

or a pharmaceutically acceptable salt, solvate, clathrate or prodrug thereof; wherein: Y, E, Z, R₁, R₂, R₃, Rₐ, R_{b}, R_{c}, R_{d}, R₄ₐ, R_{4b}, R₅ₐ, A, m, n and p are as defined above for Formula (I).

In additional embodiments, the invention is directed to a pharmaceutical composition comprising a pharmaceutically acceptable carrier or excipient and a compound of Formulae (I), (Ia) or (Ib), or a pharmaceutically acceptable salt, solvate, clathrate or prodrug thereof.

In an additional aspect, the invention is directed to a method of inhibiting deubiquitination activity of a Usp14 protein comprising contacting the Usp14 protein with a compound of Formulae (I), (Ia) or (Ib), or a pharmaceutically acceptable salt, solvate, clathrate or prodrug thereof, in an amount sufficient to inhibit deubiquitination activity of the Usp14 protein.

In yet another embodiment, the invention is directed to a method of enhancing protein degradation by a proteasome in a cell comprising contacting the cell with a compound of Formulae (I), (Ia) or (Ib), or a pharmaceutically acceptable salt, solvate, clathrate or prodrug thereof, in an amount sufficient to enhance protein degradation by the proteasome.

In additional embodiments, the invention encompasses a method of treating a patient suffering from a condition associated with a dysfunction in proteostasis comprising administering to said patient an effective amount of a compound of Formulae (I), (Ia) or (Ib).

In another aspect, the invention is directed to a method of enhancing proteasome function in a subject in need thereof comprising administering to said subject an effective amount of a compound of Formulae (I), (Ia) or (Ib), or a pharmaceutically acceptable salt, solvate, clathrate or prodrug thereof.

In a further embodiment, the invention is directed to a method for treating a condition characterized by deficient proteasome activity or deficiency of other components of the ubiquitin-proteasome pathway in a subject comprising administering to said subject an effective amount of a compound of Formulae (I), (Ia) or (Ib), or a pharmaceutically acceptable salt, solvate, clathrate or prodrug thereof.

In yet another embodiment, the invention encompasses a method of treating cancer or a tumor in a subject in need thereof comprising administering to said subject an effective amount of a compound of Formulae (I), (Ia) or (Ib), or a pharmaceutically acceptable salt, solvate, clathrate or prodrug thereof.

In a further aspect, the invention is a pharmaceutical composition comprising: a pharmaceutically acceptable carrier or excipient;

an agent selected from the group consisting of a proteostasis regulator and a pharmacologic chaperone; and

a compound of Formulae (I), (Ia) or (Ib), or a pharmaceutically acceptable salt, solvate, clathrate or prodrug thereof.

### DETAILED DESCRIPTION OF THE INVENTION

A description of preferred embodiments of the invention follows.

As used herein, the words “a” and “an” are meant to include one or more unless otherwise specified. For example, the term “a cell” encompasses both a single cell and a combination of two or more cells.

As discussed above, the present invention is directed to compounds of Formulae (I), (Ia) and (Ib), pharmaceutical compositions thereof, methods of use thereof in the treatment of conditions associated with a dysfunction in proteostasis, methods of enhancing proteasome activity and methods for treating cancer or a tumor.

In some aspects, the invention is directed to a compound having the Formula (I); or a pharmaceutically acceptable salt, solvate, clathrate or prodrug thereof.

In certain aspects, the compound has the Formula (I), or is a pharmaceutically acceptable salt, solvate, clathrate or prodrug thereof, wherein A is sulfur.

In certain additional aspects, the compound has the Formula (I), or is a pharmaceutically acceptable salt, solvate, clathrate or prodrug thereof, wherein at least one of Q₁ and Q₂ is nitrogen.

In additional embodiments, the compound has the Formula (I), or is a pharmaceutically acceptable salt, solvate, clathrate or prodrug thereof, at least one of L₃ and L₄ is C(R₆ₐ)(R_{6b}), wherein each R₆ₐ and R_{6b} are independently selected from hydrogen and C₁₋₁₀ alkyl. In another aspect, each R₆ₐ and R_{6b} are independently selected from hydrogen and C₁-C₄ alkyl.

In a further embodiment, the compound has the Formula (I), or is a pharmaceutically acceptable salt, solvate, clathrate or prodrug thereof, at least one of L₃ and L₄ is C(R₆ₐ)(R_{6b}), and wherein at least one geminal R₆ₐ and R_{6b} are taken together with the carbon atom to which they are attached to form a spiro C₃-C₁₂ cycloalkyl, a spiro C₃-C₁₂ cycloalkenyl, a spiro heterocyclic, a spiro aryl or spiro heteroaryl, each optionally substituted.

In yet another embodiment, the compound has the Formula (I), or is a pharmaceutically acceptable salt, solvate, clathrate or prodrug thereof, wherein Y is

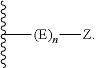

In certain aspects, each R₄ₐ and R_{4b} are independently selected from hydrogen and optionally substituted C₁-C₁₀ alkyl. In another aspect, at least one pair of R₄ₐ and R_{4b} are taken together with the carbon atom to which they are attached to form an optionally substituted optionally substituted C₃-C₁₂ cycloalkyl, optionally substituted C₃-C₁₂ cycloalkenyl, optionally substituted heterocyclic, optionally substituted aryl or optionally substituted heteroaryl.

In certain embodiments, the compound has the Formula (I), or is a pharmaceutically acceptable salt, solvate, clathrate or prodrug thereof, wherein Y is

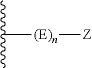

and Z is OR₃. In some embodiments, R₃ is hydrogen or optionally substituted C₁-C₁₀ alkyl.

In certain embodiments, the compound has the Formula (I), or is a pharmaceutically acceptable salt, solvate, clathrate or prodrug thereof, wherein Y is

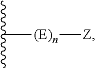

wherein Z is C(O)R₃, C(O)OR₃, C(O)NRₐR_{b}, S(O)₂NRₐR_{b} and C(O)NRₐS(O)₂R₃. In some embodiments, Z is C(O)R₃ wherein R₃ is optionally substituted C₁-C₁₀ alkyl. In another embodiment, Z is C(O)NRₐR_{b} wherein Rₐ and R_{b} are each independently hydrogen or optionally substituted C₁-C₁₀ alkyl; or alternatively, Rₐ and R_{b} are taken together with the nitrogen atom to which they are attached to form an optionally substituted heterocyclic or an optionally substituted heteroaryl.

In an additional aspect of the invention, the compound has the Formula (I), or is a pharmaceutically acceptable salt, solvate, clathrate or prodrug thereof, wherein n is 0, 1 or 2. In another aspect, n is 1.

In yet another embodiment, the compound has the Formula (I), or is a pharmaceutically acceptable salt, solvate, clathrate or prodrug thereof, wherein R₁ and R₂ are each independently selected from the group consisting of optionally substituted C₁-C₁₀ alkyl, optionally substituted C₃-C₁₂ cycloalkyl and optionally substituted heterocyclic. In an additional aspect, R₁ is optionally substituted C₁-C₁₀ alkyl and R₂ is optionally substituted C₃-C₁₂ cycloalkyl or optionally substituted heterocyclic. In yet an additional embodiment, R₁ is optionally substituted C₁-C₄ alkyl. In a further aspect, R₂ is optionally substituted C₃-C₆ cycloalkyl or optionally substituted heterocyclic. In yet a further embodiment, R₂ is optionally substituted cyclopropyl.

In additional embodiments, the compound has the Formula (Ia), or is a pharmaceutically acceptable salt, solvate, clathrate or prodrug thereof.

In yet an additional aspect, the compound has the Formula (Ia), or is a pharmaceutically acceptable salt, solvate, clathrate or prodrug thereof, wherein A is sulfur.

In another embodiment, the compound has the Formula (Ia), or is a pharmaceutically acceptable salt, solvate, clathrate or prodrug thereof, wherein m is 0 or 1.

In a further embodiment, the compound has the Formula (Ia), or is a pharmaceutically acceptable salt, solvate, clathrate or prodrug thereof, wherein L₂ is O, S(O)₂ or NR_{d}.

In an additional embodiment, the compound has the Formula (Ia), or is a pharmaceutically acceptable salt, solvate, clathrate or prodrug thereof, wherein L₁ is O, S(O)₂ or NR_{d}.

In yet an additional embodiment, the compound has the Formula (Ia), or is a pharmaceutically acceptable salt, solvate, clathrate or prodrug thereof, wherein L₂ is C(R₆ₐ)(R_{6b}). In certain aspects, L₂ is C(R₆ₐ)(R_{6b}) and L₁ is O. In certain additional aspects, L₂ is C(R₆ₐ)(R_{6b}) and L₁ is O, and m is 0 or 1.

In a further aspect, the compound has the Formula (Ia), or is a pharmaceutically acceptable salt, solvate, clathrate or prodrug thereof, wherein L₂ is C(R₆ₐ)(R_{6b}) and L₁ is S(O)₂.

In certain additional aspects, the compound has the Formula (Ia), or is a pharmaceutically acceptable salt, solvate, clathrate or prodrug thereof, wherein L₂ is C(R₆ₐ)(R_{6b}) and L₁ is NR_{d}, and m is 0 or 1.

In a further aspect, the compound has the Formula (Ia), or is a pharmaceutically acceptable salt, solvate, clathrate or prodrug thereof, wherein L₁ is S.

In yet another aspect, the compound has the Formula (Ia), or is a pharmaceutically acceptable salt, solvate, clathrate or prodrug thereof, wherein L₂ is C(R₆ₐ)(R_{6b}) and L₁ is S.

In a further embodiment, the compound has the Formula (Ia), or is a pharmaceutically acceptable salt, solvate, clathrate or prodrug thereof, wherein L₂ is C(R₆ₐ)(R_{6b}), L₁ is S and A is S.

In yet another aspect, the compound has the Formula (Ia), or is a pharmaceutically acceptable salt, solvate, clathrate or prodrug thereof, wherein A is S, L₁ is S, L₂ is C(R₆ₐ)(R_{6b}), and Y is hydrogen.

In yet an additional embodiment, the compound has the Formula (Ia), or is a pharmaceutically acceptable salt, solvate, clathrate or prodrug thereof, wherein Y is

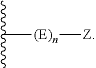

In some embodiments, each of R₄ₐ and R_{4b} are each independently selected from hydrogen and optionally substituted C₁-C₁₀ alkyl. In additional aspects, each of R₄ₐ and R_{4b} are each independently selected from hydrogen and C₁-C₃ alkyl.

In yet an additional embodiment, the compound has the Formula (Ia), or is a pharmaceutically acceptable salt, solvate, clathrate or prodrug thereof, wherein Y is

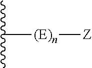

and n is 0, 1 or 2. In a further embodiment, n is 1.

In certain embodiments, the compound has the Formula (Ia), or is a pharmaceutically acceptable salt, solvate, clathrate or prodrug thereof, wherein Y is

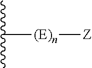

and Z is OR₃. In some embodiments, R₃ is hydrogen or optionally substituted C₁-C₁₀ alkyl.

In certain embodiments, the compound has the Formula (Ia), or is a pharmaceutically acceptable salt, solvate, clathrate or prodrug thereof, wherein Y is

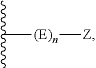

wherein Z is C(O)R₃, C(O)OR₃, C(O)NRₐR_{b}, S(O)₂NRₐR_{b}, or C(O)NRₐS(O)₂R₃. In some embodiments, Z is C(O)R₃, wherein R₃ is optionally substituted C₁-C₁₀ alkyl. In another embodiment, Z is C(O)NRₐR_{b} wherein Rₐ and R_{b} are each independently hydrogen or optionally substituted C₁-C₁₀ alkyl; or alternatively, Rₐ and R_{b} are taken together with the nitrogen atom to which they are attached to form an optionally substituted heterocyclic or an optionally substituted heteroaryl. In yet additional aspects, Z is S(O)₂NRₐR_{b}. In yet further aspects, Z is C(O)NRₐS(O)₂R₃.

In yet another embodiment, the compound has the Formula (Ia), or is a pharmaceutically acceptable salt, solvate, clathrate or prodrug thereof, wherein R₁ and R₂ are each independently selected from the group consisting of optionally substituted C₁-C₁₀ alkyl, optionally substituted C₃-C₁₂ cycloalkyl and optionally substituted heterocyclic. In an additional aspect, R₁ is optionally substituted C₁-C₁₀ alkyl and R₂ is optionally substituted C₃-C₁₂ cycloalkyl or optionally substituted heterocyclic. In yet an additional embodiment, R₁ is optionally substituted C₁-C₄ alkyl. In a further aspect, R₂ is optionally substituted C₃-C₆ cycloalkyl or optionally substituted heterocyclic. In yet a further embodiment, R₂ is optionally substituted cyclopropyl.

In additional aspects, the invention is a compound of Formula (Ib), or is a pharmaceutically acceptable salt, solvate, clathrate or prodrug thereof.

In another embodiment, the invention is a compound of Formula (Ib), or is a pharmaceutically acceptable salt, solvate, clathrate or prodrug thereof, wherein Y is

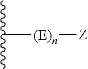

and n is 0, 1 or 2. In another embodiment, n is 1.

In yet another aspect, the invention is directed to a compound of Formula (Ib), or is a pharmaceutically acceptable salt, solvate, clathrate or prodrug thereof, wherein R₁ and R₂ are each independently selected from the group consisting of optionally substituted C₁-C₁₀ alkyl and optionally substituted C₃-C₁₂ cycloalkyl. In some embodiments, R₁ is optionally substituted C₁-C₁₀ alkyl and R₂ is optionally substituted C₃-C₁₂ cycloalkyl or optionally substituted heterocyclic. In yet additional embodiments, R₁ is optionally substituted C₁-C₄ alkyl and R₂ is optionally substituted C₃-C₆ cycloalkyl or optionally substituted heterocyclic.

In a further embodiment, R₁ is optionally substituted C₁-C₄ alkyl and R₂ is cyclopropyl.

In certain embodiments, the compound has the Formula (Ia), or a pharmaceutically acceptable salt, solvate, clathrate or prodrug thereof, wherein Y is

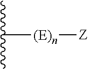

and Z is OR₃. In some embodiments, R₃ is hydrogen or optionally substituted C₁-C₁₀ alkyl.

In certain embodiments, the compound has the Formula (Ib), or is a pharmaceutically acceptable salt, solvate, clathrate or prodrug thereof, wherein Y is

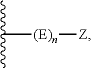

wherein Z is C(O)R₃, C(O)OR₃, C(O)NRₐR_{b}, S(O)₂NRₐR_{b}, or C(O)NRₐS(O)₂R₃. In some embodiments, Z is C(O)R₃ wherein R₃ is optionally substituted C₁-C₁₀ alkyl. In another embodiment, Z is C(O)NRₐR_{b} wherein Rₐ and R_{b} are each independently hydrogen or optionally substituted C₁-C₁₀ alkyl; or alternatively, Rₐ and R_{b} are taken together with the nitrogen atom to which they are attached to form an optionally substituted heterocyclic or an optionally substituted heteroaryl. In yet additional aspects, Z is S(O)₂NRₐR_{b}. In yet further aspects, Z is C(O)NRₐS(O)₂R₃.

In certain embodiments, the compound has the Formula (Ib), or is a pharmaceutically acceptable salt, solvate, clathrate or prodrug thereof, wherein R_{d} is selected from the group consisting of hydrogen, optionally substituted C₁-C₁₀ alkyl, C(O)R_{c}, C(O)OR_{c}, C(O)NRₐR_{b}, S(O)₂NRₐR_{b} and S(O)₂R_{c}.

In some embodiments, the compound has the Formula (Ib), or is a pharmaceutically acceptable salt, solvate, clathrate or prodrug thereof, wherein m is 1.

In other embodiments, the compound has the Formula (Ib), or is a pharmaceutically acceptable salt, solvate, clathrate or prodrug thereof, wherein m is 0.

It is to be understood that the specific embodiments described herein can be taken in combination with other specific embodiments delineated herein. For example, for compounds of Formula (I), A was defined as sulfur in certain embodiments and in certain embodiments, each of R₄ₐ and R_{4b} was described as independently selected from hydrogen and optionally substituted C₁-C₁₀ alkyl. It is thus to be understood that the invention encompasses compounds of Formula (I), wherein A is sulfur and each of R₄ₐ and R_{4b} are each independently selected from hydrogen and optionally substituted C₁-C₁₀ alkyl.

Exemplary compounds encompassed by the invention are shown in Table 1 below:

TABLE 1

| Compound | |
|---|---|
| Number | Compound |
| 1 | 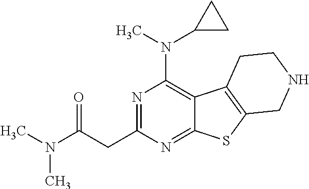 [SMILES: CN(C)C(=O)Cc1nc(N(C)C2CC2)c2c3c(sc2n1)CNCC3] |
| 2 | 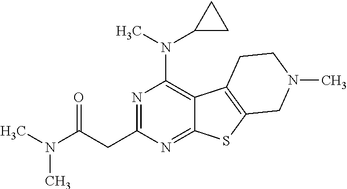 |
| 3 | 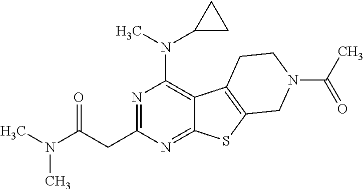 [SMILES: CC(=O)N1CCc2c(sc3nc(CC(=O)N(C)C)nc(N(C)C4CC4)c23)C1] |
| 4 | 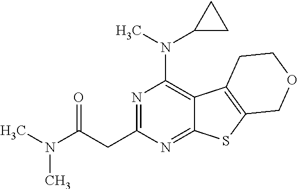 [SMILES: CN(C)C(=O)Cc1nc(N(C)C2CC2)c2c3c(sc2n1)COCC3] |
| 5 | 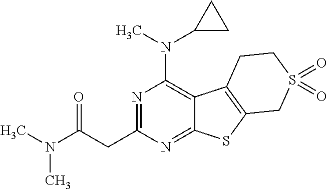 [SMILES: CN(C)C(=O)Cc1nc(N(C)C2CC2)c2c3c(sc2n1)CS(=O)(=O)CC3] |
| 6 | 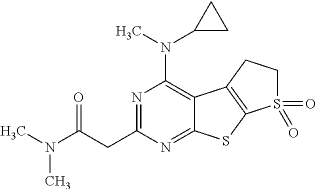 [SMILES: CN(C)C(=O)Cc1nc(N(C)C2CC2)c2c3c(sc2n1)S(=O)(=O)CC3] |
| 7 | 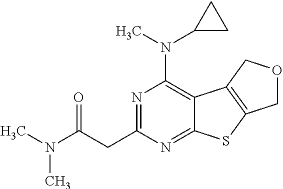 [SMILES: CN(C)C(=O)Cc1nc(N(C)C2CC2)c2c3c(sc2n1)COC3] |
| 8 | 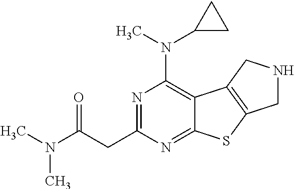 [SMILES: CN(C)C(=O)Cc1nc(N(C)C2CC2)c2c3c(sc2n1)CNC3] |
| 9 | 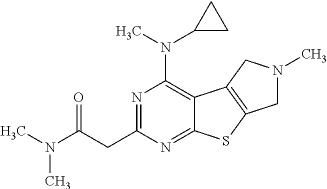 |
| 10 | 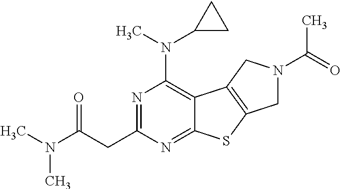 [SMILES: CC(=O)N1Cc2sc3nc(CC(=O)N(C)C)nc(N(C)C4CC4)c3c2C1] |
| 11 | 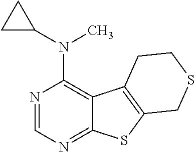 [SMILES: CN(c1ncnc2sc3c(c12)CCSC3)C1CC1] |

As used herein, the term “alkyl”, as used herein, unless otherwise indicated, refers to both branched and straight-chain saturated aliphatic hydrocarbon groups having the specified number of carbon atoms; for example, “C₁-C₁₀ alkyl” denotes alkyl having 1 to 10 carbon atoms. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term, “alkenyl”, as used herein, refers to both straight and branched-chain moieties having the specified number of carbon atoms and having at least one carbon-carbon double bond.

The term, “alkynyl”, as used herein, refers to both straight and branched-chain moieties having the specified number or carbon atoms and having at least one carbon-carbon triple bond.

The term “cycloalkyl,” as used herein, refers to monocyclic and polycyclic alkyl moieties having 3 or more carbon atoms. Polycyclic alkyl moieties include, for example, bicycloalkyl and tricycicoalkyl. Examples of cycloalkyl include, but are not limited to, cyclopropyl, cyclobutyl, cyclopentyl, cyclohexyl, cycloheptyl and adamantyl.

The term “cycloalkenyl,” as used herein, refers to monocyclic and polycyclic alkenyl moieties having 3 or more carbon atoms.

The term “cycloalkynyl,” as used herein, refers to monocyclic and polycyclic alkynyl moieties having 5 or more carbon atoms.

The term “heterocyclic” encompasses heterocycloalkyl, heterocycloalkenyl, heterobicycloalkyl, heterobicycloalkenyl, heteropolycycloalkyl, heteropolycycloalkenyl and the like. Heterocycloalkyl refers to cycloalkyl groups containing one or more heteroatoms (O, S, or N) within the ring. Heterocycloalkenyl as used herein refers to cycloalkenyl groups containing one or more heteroatoms (O, S or N) within the ring. Heterobicycloalkyl refers to bicycloalkyl groups containing one or more heteroatoms (O, S or N) within a ring. Heterobicycloalkenyl as used herein refers to bicycloalkenyl groups containing one or more heteroatoms (O, S or N) within a ring.

Cycloalkyl, cycloalkenyl, heterocyclic, groups also include groups similar to those described above for each of these respective categories, but which are substituted with one or more oxo moieties.

The term “aryl”, as used herein, refers to mono- or polycyclic aromatic carbocyclic ring systems. A polycyclic aryl is a polycyclic ring system that comprises at least one aromatic ring. Polycyclic aryls can comprise fused rings, covalently attached rings or a combination thereof. The term “aryl” embraces aromatic radicals, such as, phenyl, naphthyl, indenyl, tetrahydronaphthyl, and indanyl. An aryl group may be substituted or unsubstituted.

The term “heteroaryl”, as used herein, refers to aromatic carbocyclic groups containing one or more heteroatoms (O, S, or N) within a ring. A heteroaryl group can be monocyclic or polycyclic. A heteroaryl group may additionally be substituted or unsubstituted. The heteroaryl groups of this invention can also include ring systems substituted with one or more oxo moieties. A polycyclic heteroaryl can comprise fused rings, covalently attached rings or a combination thereof. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, thiazolopyridinyl, oxazolopyridinyl and azaindolyl. The foregoing heteroaryl groups may be C-attached or heteroatom-attached (where such is possible). For instance, a group derived from pyrrole may be pyrrol-1-yl (N-attached) or pyrrol-3-yl (C-attached).

The term “alkoxy” refers to a radical wherein an alkyl moiety is attached via an oxygen atom. Non-limiting examples of such groups include methoxy, ethoxy, propoxy, butoxy, pentoxy or hexoxy and the like.

The term “substituted” refers to substitution by independent replacement of one, two, or three or more of the hydrogen atoms with substituents including, but not limited to, —C₁-C₁₂ alkyl, —C₂-C₁₂ alkenyl, —C₂-C₁₂ alkynyl, —C₃-C₁₂ cycloalkyl, —C₃-C₁₂ cycloalkenyl, C₃-C₁₂ cycloalkynyl, -heterocyclic, —F, —Cl, —Br, —I, —OH, —NO₂, —N₃, —CN, —NH₂, oxo, thioxo, —NHR, —NRₓRₓ, dialkylamino, -diarylamino, -diheteroarylamino, —ORₓ, —C(O)R_{y}, —C(O)C(O)R_{y}, —OCO₂R_{y}, —OC(O)R_{y}, OC(O)C(O)R_{y}, —NHC(O)R_{y}, —NHCO₂R_{y}, —NHC(O)C(O)R_{y}, NHC(S)NH₂, —NHC(S)NHRₓ, —NHC(NH)NH₂, —NHC(NH)NHRₓ, —NHC(NH)Rₓ, —C(NH)NHRₓ, —NRₓC(O)Rₓ, —NRₓCO₂R_{y}, —NRₓC(O)C(O)R_{y}, —NRₓC(S)NH₂, —NRₓC(O)NRₓRₓ, —NRₓS(O)₂NRₓRₓ, —NRₓC(S)NHRₓ, —NRₓC(NH)NH₂, —NRₓC(NH)NHRₓ, —NRxC(NH)Rₓ, —C(NRx)NHRₓ—S(O)ₙR_{y}, —NHSO₂Rₓ, —CH₂NH₂, —CH₂SO₂CH₃, (C═NRₓ)Rₓ; -aryl, -arylalkyl, -heteroaryl, -heteroarylalkyl, -heterocycloalkyl, —C₃-C₁₂-cycloalkyl, -polyalkoxyalkyl, -polyalkoxy, -methoxymethoxy, -methoxyethoxy, —SH, —S—Rₓ, or -methylthiomethyl, wherein Rₓ is selected from the group consisting of —C₁-C₁₂ alkyl, —C₂-C₁₂ alkenyl, —C₂-C₁₂ alkynyl, —C₃-C₁₂ cycloalkyl, -aryl, -heteroaryl and -heterocyclic; —R_{y} is selected from the group consisting of —C₁-C₁₂ alkyl, —C₂-C₁₂ alkenyl, —C₂-C₁₂ alkynyl, —C₃-C₁₂ cycloalkyl, -aryl, -heteroaryl, -heterocyclic, —NH₂, —NH—C₁-C₁₂ alkyl, —NH—C₂-C₁₂ alkenyl, —NH—C₂-C₁₂-alkynyl, —NH—C₃-C₁₂ cycloalkyl, —NH-aryl, —NH-heteroaryl and —NH-heterocyclic, and n is 0, 1 or 2. It is understood that the aryls, heteroaryls, alkyls, and the like can be further substituted.

The term “haloalkyl” as used herein refers to an alkyl group having 1 to (2m+1) subsistent(s) independently selected from F, Cl, Br or I, where n is the maximum number of carbon atoms in the alkyl group.

Non-limiting examples of optionally substituted aryl are phenyl, substituted phenyl, napthyl and substituted naphthyl.

Certain of the compounds described herein contain one or more asymmetric centers and may thus give rise to enantiomers, diastereomers, and other stereoisomeric forms that may be defined, in terms of absolute stereochemistry, as (R)— or (S)—. The present invention is meant to include all such possible isomers, including racemic mixtures, optically pure forms and intermediate mixtures. Optically active (R)— and (S)-isomers may be prepared using chiral synthons or chiral reagents, or resolved using conventional techniques. “Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. When the compounds described herein contain olefinic double bonds or other centers of geometric asymmetry, and unless specified otherwise, it is intended that the compounds include both E and Z geometric isomers. Likewise, all tautomeric forms are also intended to be included.

Where a particular stereochemistry is described or depicted it is intended to mean that a particular enantiomer is present in excess relative to the other enantiomer. A compound has an R-configuration at a specific position when it is present in excess compared to the compound having an S-configuration at that position. A compound has an S-configuration at a specific position when it is present in excess compared to the compound having an R-configuration at that position.

Likewise, all tautomeric forms are also intended to be included. Where a particular compound is described or depicted, it is intended to encompass that chemical structure as well as tautomers of that structure.

It is to be understood that atoms making up the compounds of the present invention are intended to include isotopic forms of such atoms. Isotopes, as used herein, include those atoms having the same atomic number but different mass numbers. Isotopes of hydrogen include, for example, tritium and deuterium, and isotopes of carbon include, for example, ¹³C and ¹⁴C. The invention therefore encompasses embodiments in which one or more of the hydrogen atoms in Formula (I), (Ia), or (Ib) are replaced with deuterium. The invention also encompasses embodiments wherein one or more of the carbon atoms in Formula (I), (Ia), or (Ib) is replaced with silicon atoms.

The invention additionally encompasses embodiment wherein one or more of the nitrogen atoms in Formula (I), (Ia), or (Ib) are oxidized to N-oxide.

An exemplary synthetic route for the preparation of compounds of the invention is shown in the Scheme I below. As will be understood by the skilled artisan, diastereomers can be separated from the reaction mixture using column chromatography.

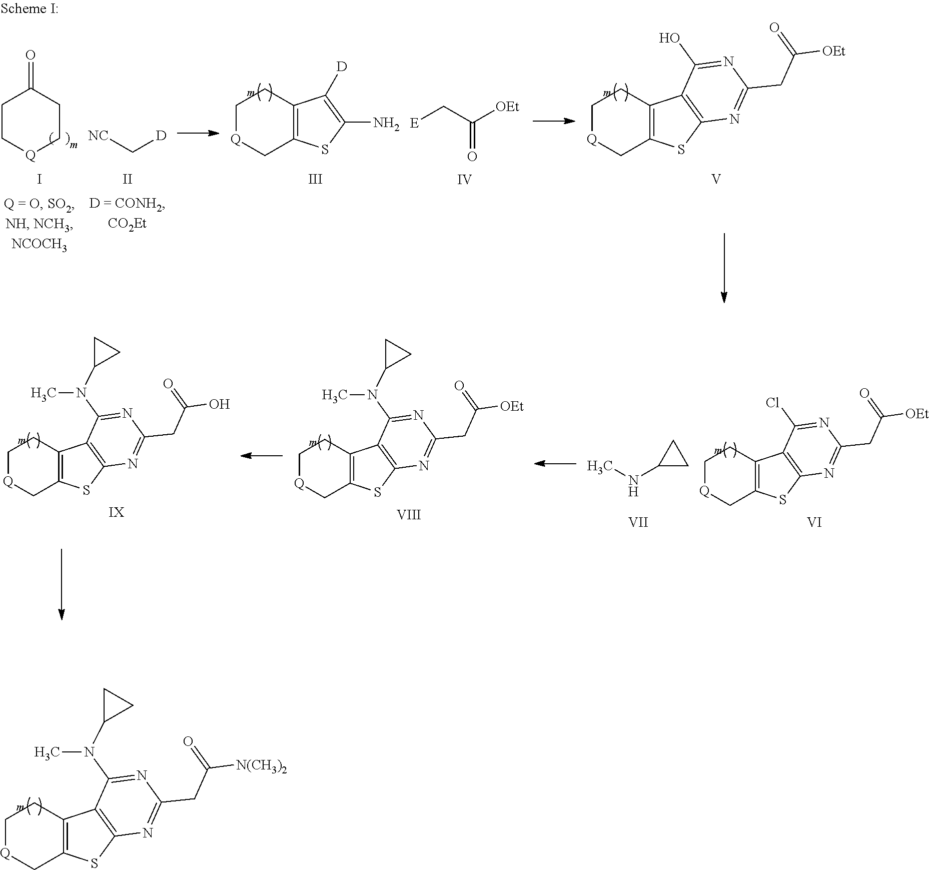

### EXAMPLE 1-10

Cycloalkyl ketone I where Q is oxygen, sulfonyl (SO₂) or nitrogen bearing a methyl, acetyl or carbamate protecting group is subjected to a Gewald Reaction with nitrile II, being either ethyl cyanoacetate where D is an ethyl ester group or cyanoacetamide where D is a carboxamide group, which affords product III. Typically the Gewald reaction is conducted in the presence of sulfur and an amine base such as triethylamine in an alcohol solvent such as ethanol at a temperature ranging from 0° C.-80° C. In the case where D in III is an ester, then E in IV is a nitrile and both agents are condensed under acidic conditions at temperatures ranging from 0° C.-50° C. to give product V, typically in a polar ether such as dioxane into which gaseous hydrogen chloride gas has been bubbled in. Alternatively, when D in III is a carboxamide, then E in IV is an ester and the condensation of both agents is conducted under basic conditions at temperatures ranging from 60° C.-100° C. to give product V, typically using an alkoxide base such as sodium ethoxide in an alcohol solvent such as ethanol. Chlorination of V to give VI is accomplished using a chlorinating reagent such as phosphorous oxychloride at temperatures ranging from 80° C.-120° C. to give product VI. Dialkylamines such as N,N-cyclopropylmethylamine VII undergo a displacement reaction with VI to give VIII, in a polar solvent such as ethanol or dimethyl sulfoxide at temperatures ranging from 0° C.-90° C. Saponification of VIII with alkali bases such as lithium or sodium hydroxide in the presence of water in solvents such as tetrahydrofuran or ethanol at temperatures ranging from 20° C.-90° C. gives product IX. A carboxamide forming reaction is utilized in converting IX into N,N-dimethylamide products represented as Compounds 1-10 where typically IX is treated with a carbodiimide coupling reagent such as EDC hydrochloride followed by an amine such as dimethylamine, in the presence of an amine base such as triethylamine, in a polar solvent such as dimethforamide at temperatures ranging from 20° C.-50° C. and optionally with a activation agent such as N-hydroxybenzotriazole. In the case of Compounds 1 and 8, the product of this reaction would be where Q is nitrogen bearing a carbamate protecting group such as a t-butylcarbamate which is subsequently removed upon treatment with an acid such as a solution of hydrogen chloride in dioxane at temperatures ranging from 20° C.-50° C. to give the amine product Compound 1 and 8 as their hydrogen chloride salt form.

The invention encompasses pharmaceutically acceptable salts of the compounds described herein. Thus, in certain aspects, the invention is directed to pharmaceutically acceptable salts of compounds of the invention and pharmaceutical compositions thereof. A “pharmaceutically acceptable salt” includes an ionic bond-containing product of the reaction between the disclosed compound with either an acid or a base, suitable for administering to a subject. Pharmaceutically acceptable salts are well known in the art and are described, for example, in Berge et al. (1977), Pharmaceutical Salts. Journal of Pharmaceutical Sciences, 69(1): 1-19, the contents of which are herein incorporated by reference. A non-limiting example of a pharmaceutically acceptable salt is an acid salt of a compound containing an amine or other basic group which can be obtained by reacting the compound with a suitable organic or inorganic acid. Examples of pharmaceutically acceptable salts also can be metallic salts including, but not limited to, sodium, magnesium, calcium, lithium and aluminum salts. Further examples of pharmaceutically acceptable salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid. Salts can also be formed with suitable organic bases when the compound comprises an acid functional group such as —C(O)OH or —SO₃H. Such bases suitable for the formation of a pharmaceutically acceptable base addition salts with compounds of the present invention include organic bases that are nontoxic and strong enough to react with the acid functional group. Such organic bases are well known in the art and include amino acids such as arginine and lysine, mono-, di-, and triethanolamine, choline, mono-, di-, and trialkylamine, such as methylamine, dimethylamine, and trimethylamine, guanidine, N-benzylphenethylamine, N-methylglucosamine, N-methylpiperazine, morpholine, ethylendiamine, tris(hydroxymethyl)aminomethane and the like.

The invention also includes hydrates of the compounds described herein, including, for example, solvates of the compounds described herein, pharmaceutical compositions comprising the solvates and methods of use of the solvates. In some embodiments, the invention is a solvate of a compound of Formula (I), (Ia), or (Ib) or a pharmaceutical composition thereof.

Also included in the present invention are prodrugs of the compounds described herein, for example, prodrugs of a compound of Formula (I), (Ia), or (Ib) or a pharmaceutical composition thereof or method of use of the prodrug.

The invention additionally includes clathrates of the compounds described herein, pharmaceutical compositions comprising the clathrates, and methods of use of the clathrates. In some embodiments, the invention is directed to clathrates of a compound of Formula (I), (Ia), or (Ib) or a pharmaceutical composition thereof.

The invention encompasses a method of inhibiting deubiquitination activity of a Usp14 protein comprising contacting the Usp14 protein with a compound described herein, or a pharmaceutically acceptable salt, solvate, clathrate or prodrug thereof, in an amount sufficient to inhibit deubiquitination activity of the Usp14 protein. In certain embodiments, a cell is contacted with the compound described herein or a pharmaceutically acceptable salt, solvate, clathrate or prodrug thereof, in an amount sufficient to inhibit deubiquitination activity of the Usp14 protein.

The invention also encompasses a method of enhancing protein degradation by a proteasome in a cell comprising contacting the cell with a compound of a compound described herein, or a pharmaceutically acceptable salt, solvate, clathrate or prodrug thereof, in an amount sufficient to enhance protein degradation by the proteasome.

As discussed above, the invention includes pharmaceutical compositions comprising a pharmaceutically acceptable carrier or excipient and a compound described herein. The compounds of Formula (I), (Ia), or (Ib), or a pharmaceutically acceptable salt, solvate, clathrate or prodrug, can be administered in pharmaceutical compositions comprising a pharmaceutically acceptable carrier or excipient. The excipient can be chosen based on the expected route of administration of the composition in therapeutic applications. The route of administration of the composition depends on the condition to be treated. For example, intravenous injection may be preferred for treatment of a systemic disorder and oral administration may be preferred to treat a gastrointestinal disorder. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

Pharmaceutical compositions comprising compounds of Formula (I), (Ia), or (Ib), or a pharmaceutically acceptable salt, solvate, clathrate or prodrug, can be administered by a variety of routes including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

The compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Injectable formulations can be prepared either as liquid solutions or suspensions; solid forms suitable for solution in, or suspension in, liquid vehicles prior to injection can also be prepared. The preparation also can also be emulsified or encapsulated in liposomes or micro particles such as polylactide, polyglycolide, or copolymer for enhanced adjuvant effect, as discussed above [Langer, Science 249: 1527, 1990 and Hanes, Advanced Drug Delivery Reviews 28: 97-119, 1997]. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, transdermal applications and ocular delivery. For suppositories, binders and carriers include, for example, polyalkylene glycols or triglycerides; such suppositories can be formed from mixtures containing the active ingredient in the range of about 0.5% to about 10%, preferably about 1% to about 2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

For the purpose of oral therapeutic administration, the pharmaceutical compositions can be incorporated with excipients and used in the form of tablets, troches, capsules, elixirs, suspensions, syrups, wafers, chewing gums and the like. Tablets, pills, capsules, troches and the like may also contain binders, excipients, disintegrating agent, lubricants, glidants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used. In another embodiment, the composition is administered as a tablet or a capsule.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like. For vaginal administration, a pharmaceutical composition may be presented as pessaries, tampons, creams, gels, pastes, foams or spray.

The pharmaceutical composition can also be administered by nasal administration. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compounds prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administered to the skin.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” will also mean to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

The invention also encompasses a method of treating a patient suffering from a condition associated with a dysfunction in protein homeostasis comprising administering to said patient a therapeutically effective amount of a compound described herein.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “subject” is an animal to be treated or in need of treatment. A “patient” is a human subject in need of treatment.

An “effective amount” refers to that amount of an agent that is sufficient to achieve a desired and/or recited effect. In the context of a therapeutic agent, an “effective amount” of the therapeutic agent that is sufficient to ameliorate of one or more symptoms of a disorder and/or prevent advancement of a disorder, cause regression of the disorder and/or to achieve a desired effect.

As used herein, the term “inhibiting” or “decreasing” encompasses causing a net decrease by either direct or indirect means. The term “increasing” or “enhancing” means to cause a net gain by either direct or indirect means.

The invention encompasses the treatment of a condition associated with a dysfunction in proteostasis. Proteostasis refers to protein homeostasis. Dysfunction in protein homeostasis is a result of protein misfolding, protein aggregation, defective protein trafficking or protein degradation. Exemplary proteins of which there can be a dysfunction in proteostasis, for example that can exist in a misfolded state, include, but are not limited to, glucocerebrosidase, hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α-N-acetylgalactosaminidase, α-neuramidase, β-glucuronidase, β-hexosamine A and acid lipase, polyglutamine, α-synuclein, Aβ peptide, tau protein, transthyretin, insulin, TAR DNA-binding protein 43 (TDP-43), ataxin-3, and rhodopsin.

In certain embodiments, the protein is selected from the group consisting of huntingtin, tau, alpha-synuclein, α1 anti-trypsin and superoxide dismutase.

Protein conformational diseases encompass gain of function disorders and loss of function disorders. In one embodiment, the protein conformational disease is a gain of function disorder. The terms “gain of function disorder,” “gain of function disease,” “gain of toxic function disorder” and “gain of toxic function disease” are used interchangeably herein. A gain of function disorder is a disease characterized by increased aggregation-associated proteotoxicity. In these diseases, aggregation exceeds clearance inside and/or outside of the cell. Gain of function diseases include, but are not limited to neurodegenerative diseases associated with aggregation of polyglutamine, Lewy body diseases, amyotrophic lateral sclerosis, transthyretin-associated aggregation diseases, Alzheimer's disease, Machado-Joseph disease, cerebral B-amyloid angiopathy, retinal ganglion cell degeneration, tautopathies (progressive supranuclear palsy, corticobasal degeration, frontotemporal lobar degeneration), cerebral hemorrhage with amyloidosis, Alexander disease, Serpinopathies, familial amyloidotic neuropathy, senile systemic amyloidosis, ApoAI amyloidosis, ApoAII amyloidosis, ApoAIV amyloidosis, familial amyloidosis of the Finnish type, lysoyzme amyloidosis, fibrinogen amyloidosis, dialysis amyloidosis, inclusion body myositis/myopathy, cataracts, medullary thyroid carcinoma, cardiac atrial amyloidosis, pituitary prolactinoma, hereditary lattice corneal dystrophy, cutaneous lichen amyloidosis, corneal lactoferrin amyloidosis, corneal lactoferrin amyloidosis, pulmonary alveolar proteinosis, odontogenic tumor amyloid, seminal vesical amyloid, sickle cell disease, critical illness myopathy, von Hippel-Lindau disease, spinocerebellar ataxia 1, Angelman syndrome, giant axon neuropathy, inclusion body myopathy with Paget disease of bone, frontotemporal dementia (IBMPFD) and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Aβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease. Prion diseases (also known as transmissible spongiform encephalopathies or TSEs) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia and Kuru.

In a further embodiment, the protein conformation disease is a loss of function disorder. The terms “loss of function disease” and “loss of function disorder” are used interchangeably herein. Loss of function diseases are a group of diseases characterized by inefficient folding of a protein resulting in excessive degradation of the protein. Loss of function diseases include, for example, lysosomal storage diseases. Lysosomal storage diseases are a group of diseases characterized by a specific lysosomal enzyme deficiency which may occur in a variety of tissues, resulting in the build-up of molecules normally degraded by the deficient enzyme. The lysosomal enzyme deficiency can be in a lysosomal hydrolase or a protein involved in the lysosomal trafficking. Lysosomal storage diseases include, but are not limited to, aspartylglucosaminuria, Fabry's disease, Batten disease, Cystinosis, Farber, Fucosidosis, Galactasidosialidosis, Gaucher's disease (including Types 1, 2 and 3), Gml gangliosidosis, Hunter's disease, Hurler-Scheie's disease, Krabbe's disease, α-Mannosidosis, β-Mannosidosis, Maroteaux-Lamy's disease, Metachromatic Leukodystrophy, Morquio A syndrome, Morquio B syndrome, Mucolipidosis II, Mucolipidosis III, Neimann-Pick Disease (including Types A, B and C), Pompe's disease, Sandhoff disease, Sanfilippo syndrome (including Types A, B, C and D), Schindler disease, Schindler-Kanzaki disease, Sialidosis, Sly syndrome, Tay-Sach's disease and Wolman disease.

In another embodiment, the disease associated with a dysfunction in proteostasis is a cardiovascular disease. Cardiovascular diseases include, but are not limited to, coronary artery disease, myocardial infarction, stroke, restenosis and arteriosclerosis. Conditions associated with a dysfunction of proteostasis also include ischemic conditions, such as, ischemia/reperfusion injury, myocardial ischemia, stable angina, unstable angina, stroke, ischemic heart disease and cerebral ischemia.

In yet another embodiment, the disease associated with a dysfunction in proteostasis is diabetes and/or complications of diabetes, including, but not limited to, diabetic retinopathy, cardiomyopathy, neuropathy, nephropathy, and impaired wound healing.

In a further embodiment, the disease associated with a dysfunction in proteostasis is an ocular disease including, but not limited to, age-related macular degeneration (AMD), diabetic macular edema (DME), diabetic retinopathy, glaucoma, cataracts, retinitis pigmentosa (RP) and dry macular degeneration.

In some embodiments, the condition associated with a dysfunction in proteostasis is selected from the group consisting of Huntington's disease, Alzheimer's disease, Parkinson's disease, amyotrophic lateral sclerosis, diabetes and complications thereof, ocular diseases and cancer or tumor.

Additional conditions associated with a dysfunction of proteostasis include hemoglobinopathies, inflammatory diseases, intermediate filament diseases, drug-induced lung damage and hearing loss. The invention also encompasses methods for the treatment of hemoglobinopathies (such as sickle cell anemia), an inflammatory disease (such as inflammatory bowel disease, colitis, ankylosing spondylitis), intermediate filament diseases (such as non-alcoholic and alcoholic fatty liver disease) and drug induced lung damage (such as methotrexate-induced lung damage). The invention additionally encompasses methods for treating hearing loss, such as noise-induced hearing loss, aminoglycoside-induced hearing loss, and cisplatin-induced hearing loss.

In addition to conditions associated with a dysfunction in proteostasis, the compound of the present invention can be used to treat a disease or condition characterized by deficient proteasome activity or deficient activity of other components of the ubiquitin-proteasome pathway. Such conditions include, for example, Hippel-Lindau disease, spino-cerebellar ataxia 1, Angelman syndrome, giant axon neuropathy, inclusion body myopathy with Paget disease, and frontotemporal dementia.

In certain embodiments, the invention encompasses a method for the treatment of a condition selected from the group consisting of Parkinson's disease, Alzheimer's disease, Frontotemporal lobar dementia (FTLD), Progressive Supranuclear Palsy (PSP), Amyotrophic lateral sclerosis (ALS), Spinocerebellar ataxia (SCA), Retinitis pigmentosum, prion diseases and autism.

In certain embodiments, the invention includes methods for the treatment of a condition associated with a dysfunction in proteostasis comprising administering to a patient in need thereof an effective amount of a compound of Formula (I), (Ia) or (Ib), or a pharmaceutically acceptable salt, solvate, clathrate or prodrug, or the compounds described herein, and a second agent (e.g., a second therapeutic agent). Co-administered agents, compounds, or therapeutics need not be administered at exactly the same time. In certain embodiments, however, the compound encompassed by Formula (I), (Ia), or (Ib), or a pharmaceutically acceptable salt, solvate, clathrate or prodrug, or a compound described herein, is administered substantially simultaneously as the second agent. By “substantially simultaneously,” it is meant that the compound of Formula (I), (Ia), or (Ib), or a pharmaceutically acceptable salt, solvate, clathrate or prodrug, or a compound described herein, is administered before, at the same time, and/or after the administration of the second agent, and encompasses, for example, administration within the same treatment session or as part of the same treatment regimen. Exemplary second agents include pharmacologic chaperones and proteostasis regulators (such as, those described below).

In yet additional aspects, the invention encompasses a method for treating a condition characterized by deficient proteasome activity or deficiency of other components of the ubiquitin-proteasome pathway in a subject comprising administering to said subject an effective amount of a compound of the invention, or a pharmaceutically acceptable salt, solvate, clathrate or prodrug thereof.

In an additional embodiment, the invention is directed to a pharmaceutical composition comprising a compound of Formula (I), (Ia), or (Ib), or a pharmaceutically acceptable salt, solvate, clathrate or prodrug, and a second agent, wherein the second agent is selected from the group consisting of a pharmacologic chaperone and a proteostasis regulator. The invention also encompasses a method of treating a patient suffering from a condition associated with a dysfunction in proteostasis comprising administering a therapeutically effective amount of a compound of the invention and a second agent, wherein the second agent is a pharmacologic chaperone. Pharmacologic chaperones or kinetic stabilizers refer to compounds that bind an existing steady state level of the folded mutant protein and chemically enhance the folding equilibrium by stabilizing the fold [Bouvier, Chem Biol 14: 241-242, 2007; Fan et al., Nat Med 5: 112-115, 1999; Sawkar et al., Proc Natl Acad Sci USA 99:15428-15433, 2002; Johnson and Kelly, Accounts of Chemical Research 38: 911-921, 2005]. The pharmacologic chaperone is administered in amount that in combination with a compound described herein in an amount that is sufficient to treat a patient suffering from a condition associated with a dysfunction in proteostasis. Exemplary pharmacologic chaperones are described in U.S. Patent Application Publication No's. 20080056994, 20080009516, 20070281975, 20050130972, 20050137223, 20050203019, 20060264467 and 20060287358, the contents of each of which are incorporated by reference herein.

In another embodiment, the invention is a method of treating a patient suffering from a condition associated with a dysfunction in proteostasis comprising administering to said patient an effective amount of a compound described herein and a second agent, wherein the second agent is a proteostasis regulator. The term “proteostasis regulator” refers to small molecules, siRNA and biologicals (including, for example, proteins) that enhance cellular protein homeostasis. For example, proteostasis regulators can be agents that influence protein synthesis, folding, trafficking and degradation pathways. Proteostasis regulators encompass pharmacologic agents that stimulate the heat shock response (HSR) signaling activity. Proteostasis regulators function by manipulating signaling pathways, including, but not limited to, the heat shock response or the unfolded protein response, or both, resulting in transcription and translation of proteostasis network components. Proteostasis regulators can enhance the folding, trafficking and function of proteins (for example, mutated proteins). Proteostasis regulators can also regulate protein chaperones by upregulating transcription or translation of the protein chaperone, or inhibiting degradation of the protein chaperone. Proteostasis regulators can influence the biology of folding, often by the coordinated increase in chaperone and folding enzyme levels and macromolecules that bind to partially folded conformational ensembles, thus enabling their progression to intermediates with more native structure and ultimately increasing the concentration of folded mutant protein for export. In one aspect, the proteostasis regulator is distinct from a chaperone in that the proteostasis regulator can enhance the homeostasis of a mutated protein but does not bind the mutated protein. In addition, proteostasis regulators can upregulate an aggregation pathway or a disaggregase activity. Exemplary proteostasis regulators are the celastrols, MG-132 and L-type Ca²⁺ channel blockers (e.g., dilitiazem and verapamil). The term “celastrols” refers to celastrol and derivatives or analogs thereof, including, but not limited to, those celastrol derivatives described in Westerheide et al., J Biol Chem, 2004. 279(53): p. 56053-60, the contents of which are expressly incorporated by reference herein. Celastrol derivatives include, for example, celastrol methyl ester, dihydrocelastrol diacetate, celastrol butyl ether, dihydrocelastrol, celastrol benzyl ester, primesterol, primesterol diacetate and triacetate of celastrol. In certain aspects, the proteostasis regulator is a heat shock response activator. A heat shock response activator is an agent that indirectly or directly activates the heat shock response, for example, by directly or indirectly activating heat shock transcription factor 1 (HSF1), inhibiting Hsp90, and/or activating chaperone expression (Westerheide et al., J Biol Chem, 2004. 279(53): p. 56053-60, the contents of which are expressly incorporated by reference herein). The terms “heat shock response activator,” “heat shock activator,” “heat shock response inducer,” and “heat shock inducer” are used interchangeably herein. Non-limiting examples of heat shock response activators are celastrols, non-steroidal anti-inflammatory drugs, ansamycin, geldenamycin, radiciol, glucuronic acid, and tributylin. Heat shock response activators have also been described, for example, in U.S. Patent Application Publication No's. 20070259820, 20070207992, 20070179087, 20060148767, the contents of each of which are expressly incorporated by reference herein. In some embodiments, the heat shock response activator is a small molecule heat shock response activator.

The invention also encompasses a method of treating cancer or a tumor in a patient in need thereof comprising administering to said patient an effective amount of a compound of Formula (I), (Ia), or (Ib). The invention additionally encompasses a method of treating cancer or a tumor in a patient in need thereof comprising administering to said patient an effective amount of a compound described herein. Cancers that can be treated according to methods of the present invention include, but are not limited to, breast cancer, colon cancer, pancreatic cancer, prostate cancer, lung cancer, ovarian cancer, cervical cancer, multiple myeloma, basal cell carcinoma, neuroblastoma, hematologic cancer, rhabdomyosarcoma, liver cancer, skin cancer, leukemia, basal cell carcinoma, bladder cancer, endometrial cancer, glioma, lymphoma, and gastrointestinal cancer.

In another embodiment, the invention is a method of treating cancer or a tumor comprising administering a compound of Formula (I), (Ia), or (Ib) or a compound described herein in combination with the administration of a chemotherapeutic agent. Chemotherapeutic agents that can be utilized include, but are not limited to, alkylating agents such as cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

In a further embodiment, the invention is a method of treating cancer or a tumor comprising administering to a patient in need thereof an effective amount of a compound of Formula (I), (Ia), or (Ib) or a compound described herein in combination with radiation therapy.

In yet an additional embodiment, the invention is a method of treating a viral infection comprising administering to a subject in need thereof an effective amount of a compound of Formula (I), (Ia), or (Ib), or a compound described herein. In certain embodiments, the viral infection is an infection from a virus of the flavivirus family. Examples of viruses in the flavivirus family include, for example, Dengue virus, West Nile virus, Japanese encephalitis virus, yellow fever virus and tick-borne encephalitis viruses. In an additional embodiment, the virus is the La Crosse virus. In another embodiment, the virus is Dengue virus or West Nile virus.

The invention is illustrated by the following examples which are not meant to be limiting in any way.

### EXEMPLIFICATION

### Example 1

### Preparation of Compounds 1-10

Referring to Scheme 1 above, cycloalkyl ketone I where Q is oxygen, sulfonyl (SO₂) or nitrogen bearing a methyl, acetyl or carbamate protecting group is subjected to a Gewald Reaction with nitrile II, being either ethyl cyanoacetate where D is an ethyl ester group or cyanoacetamide where D is a carboxamide group, which affords product III. Typically the Gewald reaction is conducted in the presence of sulfur and an amine base such as triethylamine in an alcohol solvent such as ethanol at a temperature ranging from 0° C.-80° C. In the case where D in III is an ester, then E in IV is a nitrile and both agents are condensed under acidic conditions at temperatures ranging from 0° C.-50° C. to give product V, typically in a polar ether such as dioxane into which gaseous hydrogen chloride gas has been bubbled in. Alternatively, when D in III is a carboxamide, then E in IV is an ester and the condensation of both agents is conducted under basic conditions at temperatures ranging from 60° C.-100° C. to give product V, typically using an alkoxide base such as sodium ethoxide in an alcohol solvent such as ethanol. Chlorination of V to give VI is accomplished using a chlorinating reagent such as phosphorous oxychloride at temperatures ranging from 80° C.-120° C. to give product VI. Dialkylamines such as N,N-cyclopropylmethylamine VII undergo a displacement reaction with VI to give VIII, in a polar solvent such as ethanol or dimethyl sulfoxide at temperatures ranging from 0° C.-90° C. Saponification of VIII with alkali bases such as lithium or sodium hydroxide in the presence of water in solvents such as tetrahydrofuran or ethanol at temperatures ranging from 20° C.-90° C. gives product IX. A carboxamide forming reaction is utilized in converting IX into N,N-dimethylamide products represented as Compounds 1-10 where typically IX is treated with a carbodiimide coupling reagent such as EDC hydrochloride followed by an amine such as dimethylamine, in the presence of an amine base such as triethylamine, in a polar solvent such as dimethforamide at temperatures ranging from 20° C.-50° C. and optionally with a activation agent such as N-hydroxybenzotriazole. In the case of Compounds 1 and 8, the product of this reaction would be where Q is nitrogen bearing a carbamate protecting group such as a t-butylcarbamate which is subsequently removed upon treatment with an acid such as a solution of hydrogen chloride in dioxane at temperatures ranging from 20° C.-50° C. to give the amine product Compound 1 and 8 as their hydrogen chloride salt form.

Using previously described methodology (B. H. Lee et al. Nature 2010, 467 (9), 179; the contents of which are expressly incorporated by reference herein), select compounds described herein were found to inhibit USP14. Known USP14 inhibitor IU1 (1-(1-(4-fluorophenyl)-2,5-dimethyl-1H-pyrrol-3-yl)-2-(pyrrolidin-1-yl)ethanone; B. H. Lee et al. Nature 2010, 467 (9), 179) was used as a comparative reference agent.

### Example 2

### Preparation of N-cyclopropyl-N-methyl-6,8-dihydro-5H-thiopyrano[4′,3′:4,5]thieno[2,3-dlpyrimidin-4-amine (Compound 11)

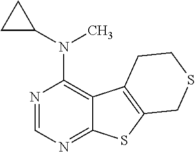 [SMILES: CN(c1ncnc2sc3c(c12)CCSC3)C1CC1]

The POCl₃ (1.3 ml, 13.37 mmol) was added dropwise to a stirred suspension of 3,5,6,8-tetrahydro-4H-thiopyrano[4′,3′:4,5]thieno[2,3-d]pyrimidin-4-one (300 mg, 1.34 mmol) in toluene (15 ml). The reaction mixture was heated at 100° C. for 18 hr, then cooled and concentrated under vacuum. Toluene (3×20 ml) was added, and the solvent removed under vacuum. The residue was slowly diluted with 5 ml H₂O then poured into 5% NaHCO₃ (100 ml) solution, and extracted with EtOAc (2×100 ml). The combined organic extracts were washed with H₂O (1×20 ml), brine (1×20 ml), dried (MgSO4) and concentrated to give a solid. The crude was purified by chromatography on silica gel eluting with 35% EtOAc/Hexanes to give 215 mg of 4-chloro-6,8-dihydro-5H-thiopyrano[4′,3′:4,5]thieno[2,3-d]pyrimidine as an off white solid. ¹H NMR (300 MHz, CDCl₃) δ ppm=2.99-3.16 (m, 2H); 3.40-3.56 (m, 2H); 3.88 (s, 2H); 8.76 (s, 1H). HPLC-MS [M, M+2]: 242.87, 244.89.

A mixture of 4-chloro-6,8-dihydro-5H-thiopyrano[4′,3′:4,5]thieno[2,3-d]pyrimidine (205 mg, 0.84 mmol), di-isopropylethylamine (0.44 ml, 2.53 mmol) and N-methylcyclopropanamine-HCl (136 mg, 1.27 mmol) were placed in a vial, and n-BuOH (10 ml) was added. The vial was sealed, and the stirred mixture was heated at 60° C. for 18 hr. The reaction mixture was concentrated under vacuum. The residue was dissolved in 200 ml EtOAc then sequentially washed with saturated NaHCO₃ solution (1×20 ml), H₂O (1×20 ml), and brine (1×20 ml), then dried (MgSO₄) and concentrated to give an oil. The crude was purified by chromatography on silica gel using 40% EtOAc/Hexanes to give an oil. The oil was dissolved in hot hexanes and precipitated upon cooling to give 131 mg of the desired product as fine white solids. ¹H NMR (300 MHz, CDCl₃) δ ppm=0.46-0.48 (m, 2H); 0.78-0.81 (m, 2H); 2.82-2.89 (m, 3H); 3.04 (s, 3H); 3.24-3.27 (m, 2H); 3.95 (s, 2H); 8.54 (s, 1H). HPLC-MS [M+1]: 277.95.

While this invention has been particularly shown and described with references to preferred embodiments thereof, it will be understood by those skilled in the art that various changes in form and details may be made therein without departing from the scope of the invention encompassed by the appended claims.

1. A compound having the Formula (I): 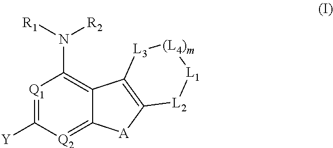
or a pharmaceutically acceptable salt, solvate, clathrate or prodrug thereof; wherein:
Q₁ and Q₂ are each independently selected from the group consisting of nitrogen and CR₆ₐ;
A is sulfur, oxygen or NR₅ₐ;
R₁ and R₂ are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₁-C₁₀ alkoxy, optionally substituted C₃-C₁₂ cycloalkyl, optionally substituted C₃-C₁₂ cycloalkenyl, optionally substituted heterocyclic, optionally substituted aryl and optionally substituted heteroaryl; or alternatively, R₁ and R₂ are taken together with the nitrogen atom to which they are attached to form an optionally substituted heterocylic or an optionally substituted heteroaryl;
Y is selected from the group consisting of hydrogen and 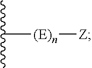
E is C(R₄ₐ)(R_{4b});
Z is selected from the group consisting of hydrogen, optionally substituted C₁-C₁₀ alkyl, optionally substituted C₂-C₁₀ alkenyl, optionally substituted C₂-C₁₀ alkynyl, OR₃, C(O)R₃, C(O)OR₃, C(O)NRₐS(O)₂R₃, OC(O)R₃, C(O)NRₐR_{b}, S(O)₂NRₐR_{b}, S(O)NRₐR_{b}, NRₐS(O)₂R₃, CN, SR₃, S(O)R₃, S(O)₂R₃, P(O)(OR₃)₂, NRₐR_{b}, N(Rₐ)OR₃, NRₐC(O)C(O)R₃, NRₐC(O)R₃, NRₐC(O)NRₐR_{b}, NRₐS(O)₂NRₐR_{b}, optionally substituted C₃-C₁₂ cycloalkyl, optionally substituted C₃-C₁₂ cycloalkenyl, optionally substituted heterocyclic, optionally substituted aryl and optionally substituted heteroaryl;
R₃ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;
Rₐ and R_{b} are each independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; alternatively, Rₐ and R_{b} are taken together with the nitrogen atom to which they are attached to form an optionally substituted heterocyclic or an optionally substituted heteroaryl;
L₁, L₂, L₃ and L₄ are each independently selected from C(R₆ₐ)(R_{6b}), O, NR_{d,} S, S(O), and SO₂, wherein at least one of L₁, L₂, L₃ and L₄ is O, NR_{d,} S, S(O) and SO₂;
each of R₄ₐ and R_{4b} are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{c}, SR_{c}, NRₐR_{b}, C(O)OR_{c}, NO₂, CN, C(O)R_{c}, C(O)C(O)R_{c}, C(O)NRₐR_{b}, NRₐC(O)R_{c}, NRₐS(O)ₚR_{c}, N(Rₐ)C(O)OR_{c}, NRₐC(O)C(O)R_{c}, NRₐC(O)NRₐR_{b}, NRₐS(O)ₚNRₐR_{b}, S(O)ₚR_{c}, S(O)ₚNRₐR_{b}, OC(O)OR_{c}, and (C═NRₐ)R_{c}; alternatively, R₄ₐ and R_{4b} can be taken together with the carbon atom to which they are attached to form an optionally substituted optionally substituted C₃-C₁₂ cycloalkyl, optionally substituted C₃-C₁₂ cycloalkenyl, optionally substituted heterocyclic, optionally substituted aryl or optionally substituted heteroaryl;
R₅ₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;
each of R₆ₐ and R_{6b} are, at each occurrence, independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{c}, SR_{c}, NRₐR_{b}, C(O)OR_{c}, NO₂, CN, C(O)R_{c}, C(O)C(O)R_{c}, C(O)NRₐR_{b}, C(O)NRₐS(O)₂R₃, NRₐC(O)R_{c}, NRₐS(O)ₚR_{c}, N(Rₐ)C(O)OR_{c}, NRₐC(O)C(O)R_{c}, NRₐC(O)NRₐR_{b}, NRₐS(O)ₚNRₐR_{b}, S(O)ₚR_{c}, S(O)ₚNRₐR_{b}, OC(O)OR_{c}, and (C═NRₐ)R_{c}; alternatively, geminal R₆ₐ and R_{6b} can be taken together with the carbon atom to which they are attached to form a spiro C₃-C₁₂ cycloalkyl, a spiro C₃-C₁₂ cycloalkenyl, a spiro heterocyclic, a spiro aryl or spiro heteroaryl, each optionally substituted;
each R_{c} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;
each R_{d} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, C(O)R_{c}, C(O)C(O)R_{c}, C(O)OR_{c}, C(O)NRₐR_{b}, S(O)ₚR_{c}, and S(O)ₚNRₐR_{b};
m and n are each independently selected from the group consisting of 0, 1, 2 and 3; and
p is 1 or 2.

2. The compound of claim 1, wherein A is sulfur.

3. The compound of claim 1, wherein at least one of Q₁ and Q₂ is nitrogen.

4. The compound of claim 1, wherein at least one of L₃ and L₄ is C(R₆ₐ)(R_{6b}), wherein each R₆ₐ and R_{6b} are independently selected from hydrogen and C₁₋₁₀ alkyl.

5. (canceled)

6. (canceled)

7. The compound of claim 1, wherein Y is

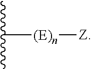

8. The compound of claim 7, wherein each R₄ₐ and R_{4b} are independently selected from hydrogen and optionally substituted C₁-C₁₀ alkyl.

9. (canceled)

10. The compound of claim 7, wherein Z is OR₃, C(O)R₃, C(O)OR₃, C(O)NRₐR_{b,} S(O)₂NRₐR_{b}, and C(O)NRₐS(O)₂R₃.

11. The compound of claim 10, wherein Z is OR₃ and R₃ is hydrogen or optionally substituted C₁-C₁₀ alkyl.

12. (canceled)

13. (canceled)

14. The compound of claim 10, wherein Z is C(O)R₃ and R₃ is optionally substituted C₁-C₁₀ alkyl.

15. (canceled)

16. The compound of claim 1, wherein n is 0, 1 or 2.

17. (canceled)

18. The compound of claim 1 having the Formula (Ia):

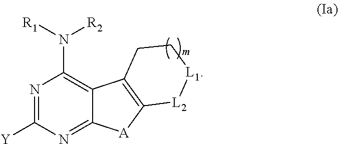

19. The compound of claim 18, wherein A is sulfur.

20. The compound of claim 18, wherein m is 0 or 1.

21. The compound of claim 18, wherein L₂ is O, S(O)₂ or NR_{d}.

22. The compound of claim 18, wherein L₁ is O, S(O)₂ or NR_{d}.

23. The compound of claim 18, wherein L₂ is C(R₆ₐ)(R_{6b}).

24-32. (canceled)

33. The compound of claim 18, wherein Y is

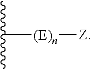

34. The compound of claim 33, wherein each of R₄ₐ and R_{4b} are each independently selected from hydrogen and optionally substituted C₁-C₁₀ alkyl.

35. (canceled)

36. The compound of claim 33, wherein n is 0, 1 or 2.

37. (canceled)

38. The compound of claim 1, wherein R₁ and R₂ are each independently selected from the group consisting of optionally substituted C₁-C₁₀ alkyl, optionally substituted C₃-C₁₂ cycloalkyl and optionally substituted heterocyclic.

39. The compound of claim 38, wherein R₁ is optionally substituted C₁-C₁₀ alkyl and R₂ is optionally substituted C₃-C₁₂ cycloalkyl or optionally substituted heterocyclic.

40. (canceled)

41. The compound of claim 39, wherein R₂ is optionally substituted C₃-C₆ cycloalkyl or optionally substituted heterocyclic.

42. The compound of claim 41, wherein R₂ is optionally substituted cyclopropyl.

43. The compound of claim 33, wherein Z is OR₃, C(O)R₃, C(O)OR₃, C(O)NRₐR_{b}, S(O)₂NRₐR_{b} or C(O)NRₐS(O)₂R₃.

44-50. (canceled)

51. The compound of claim 23 selected from the following:

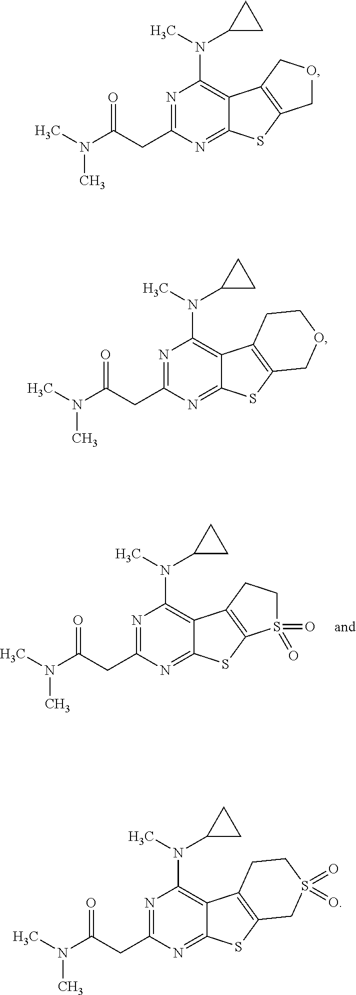 [SMILES: CN(C)C(=O)Cc1nc(N(C)C2CC2)c2c3c(sc2n1)COC3 ; CN(C)C(=O)Cc1nc(N(C)C2CC2)c2c3c(sc2n1)COCC3 ; CN(C)C(=O)Cc1nc(N(C)C2CC2)c2c3c(sc2n1)S(=O)(=O)CC3 ; "and" ; CN(C)C(=O)Cc1nc(N(C)C2CC2)c2c3c(sc2n1)CS(=O)(=O)CC3]

52. (canceled)

53. The compound of claim 1 having the Formula (Ib):

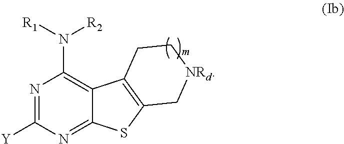

54. The compound of claim 53, wherein Y is 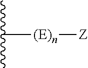
and n is 0, 1 or 2.

55. The compound of claim 54, wherein n is 1.

56. The compound of claim 53, wherein R₁ and R₂ are each independently selected from the group consisting of optionally substituted C₁-C₁₀ alkyl and optionally substituted C₃-C₁₂ cycloalkyl.

57. The compound of claim 56, wherein R₁ is optionally substituted C₁-C₁₀ alkyl and R₂ is optionally substituted C₃-C₁₂ cycloalkyl or optionally substituted heterocyclic.

58. (canceled)

59. The compound of claim 57, wherein R₁ is optionally substituted C₁-C₄ alkyl and R₂ is cyclopropyl.

60. The compound of claim 52, wherein Z is OR₃, C(O)R₃, C(O)OR₃, C(O)NRₐR_{b}, S(O)₂NRₐR_{b} or C(O)NRₐS(O)₂R₃.

61-69. (canceled)

70. The compound of claim 53, wherein R_{d} is selected from the group consisting of hydrogen, optionally substituted C₁-C₁₀ alkyl, C(O)R_{c}, C(O)OR_{c}, C(O)NRₐR_{b}, S(O)₂NRₐR_{b} and S(O)₂R_{c}.

71. The compound of claim 53, wherein m is 1.

72. The compound of claim 53, wherein m is 0.

73. The compound of claim 71 selected from the following:

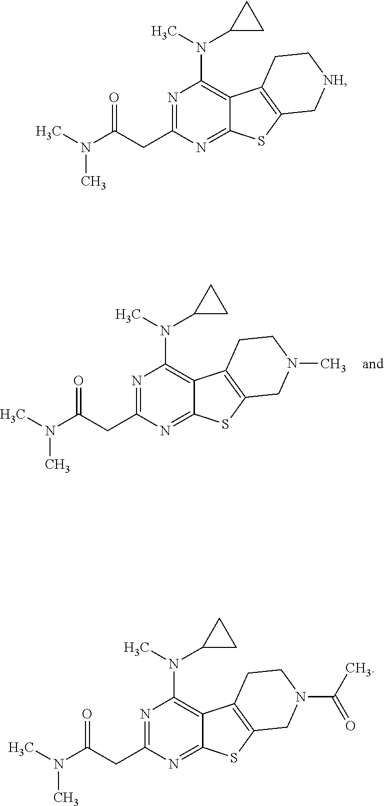

74. The compound of claim 72 selected from the following:

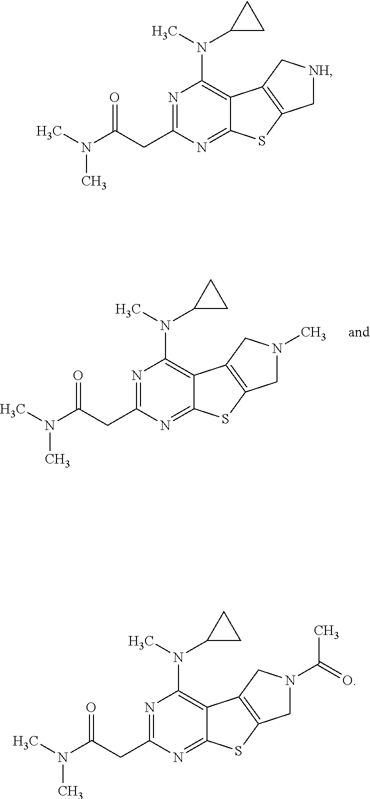

75. (canceled)

76. The compound of claim 23 having the chemical structure:

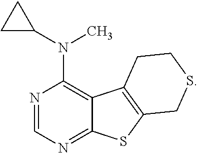 [SMILES: CN(c1ncnc2sc3c(c12)CCSC3)C1CC1]

77. A pharmaceutical composition comprising a pharmaceutically acceptable carrier or excipient and a compound of claim 1, or a pharmaceutically acceptable salt, solvate, clathrate or prodrug thereof.

78. (canceled)

79. (canceled)

80. A method of treating a patient suffering from a condition associated with a dysfunction in proteostasis comprising administering to said patient an effective amount of a compound of claim 1, or a pharmaceutically acceptable salt, solvate, clathrate or prodrug thereof.

81. (canceled)

82. (canceled)

83. The method of claim 80, wherein the condition is associated with a dysfunction in the proteostasis of a protein selected from the group consisting of hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α1 anti-trypsin, α-N-acetylgalactosaminidase, α-neuramidase, β-glucuronidase, β-hexosamine A and acid lipase, polyglutamine, α-synuclein, Aβ peptide, tau protein, hERG potassium channel, islet amyloid polypeptide, transthyretin, Huntingtin, superoxide dismutase, TAR DNA-binding protein 43 (TDP-43), and ataxin-3, rhodopsin.

84. (canceled)

85. (canceled)

86. The method of claim 80, wherein the condition is selected from the group consisting of Parkinson's disease, Alzheimer's disease, Frontotemporal lobar dementia (FTLD), Progressive Supranuclear Palsy (PSP), Amyotrophic lateral sclerosis (ALS), Spinocerebellar ataxia (SCA), Retinitis pigmentosum, Prion diseases autism, Huntington's disease, diabetes and complications of diabetes.

87-92. (canceled)
